(19) United States
(12) Patent Application Publication
(10) Pub. No.: US 2023/0102812 A1
(43) Pub. Date: Mar. 30, 2023

(54) BIOGAS-UTILIZING METHANATION SYSTEM

(71) Applicant: MITSUBISHI HEAVY INDUSTRIES, LTD., Tokyo (JP)

(72) Inventors: Taiji Yoshida, Yokohama-shi (JP); Mitsuyoshi Iwata, Yokohama-shi (JP); Nagao Hisatome, Yokohama-shi (JP)

(73) Assignee: MITSUBISHI HEAVY INDUSTRIES, LTD., Tokyo (JP)

(21) Appl. No.: 17/911,450

(22) PCT Filed: Jan. 28, 2021
(86) PCT No.: PCT/JP2021/002949

§ 371 Date: Sep. 14, 2022

(30) Foreign Application Priority Data
Mar. 27, 2020 (JP) 2020-058488

(51) Int. Cl. H01M 8/0612; C07C 1/12; H01M 8/0438; H01M 8/04537; H01M 8/04746; H01M 8/04858; H01M 8/0668

(52) U.S. Cl. CPC H01M 8/0612; C07C 1/12; H01M 8/0438; H01M 8/04604; H01M 8/04746; H01M 8/04925; H01M 8/0668; H01M 2250/10; H01M 2250/402

(57) ABSTRACT

A biogas-utilizing methanation system includes: a solid oxide fuel cell using a to-be-treated gas as a fuel gas; a hydrogen production device capable of producing hydrogen by using power of a renewable energy power generation device; and a methanation device capable of methanating carbon dioxide in the system with the hydrogen produced by the hydrogen production device. The carbon dioxide in the system can be stored in a storage device on the basis of the supply amount of the to-be-treated gas or the power of the renewable energy power generation device.

### TECHNICAL FIELD

The present disclosure relates to a methanation system utilizing biogas.

### BACKGROUND

Biogas, such as food residue gas discharged from food plants and digestion gas discharged from sewage treatment facilities, contains combustible substances such as methane and is attracting attention as a new fuel source. This type of biogas has conventionally been used as fuel for thermal power generation devices using boilers and gas engines, or as fuel for phosphoric acid fuel cells (PAFCs) after reforming, but the power generation efficiency is relatively low (for example, 20 to 40%), and it is expected to improve efficiency. Further, since biogas itself and exhaust fuel gas generated by combustion of biogas contain carbon dioxide, which causes a greenhouse effect, it is required to suppress carbon dioxide emissions into the atmosphere.

In response to such problems, for example, Patent Document 1 proposes an energy system capable of improving energy efficiency and suppressing carbon dioxide emissions by combining a water electrolysis device and a methanation device with a cogeneration system. In this system, hydrogen produced by the water electrolysis device is used in the methanation device to react carbon dioxide contained in biogas or carbon dioxide contained in exhaust fuel gas from the cogeneration system to produce methane, and the methane is supplied to an energy load network, in order to suppress carbon dioxide emissions to the outside. It is also mentioned that the water electrolysis device uses electric power of a renewable energy power generation system to contribute to reducing the environmental impacts.

### CITATION LIST

### Patent Literature

Patent Document 1: JP2019-90084A

### SUMMARY

### Problems to be Solved

Biomass emissions can vary greatly with the seasons. For example, biogas discharged from beer factories increases in summer and winter seasons when beer consumption increases, and decreases in other seasons when beer consumption decreases. Since such variations in biomass emissions are not taken into consideration in Patent Document 1, depending on the operational condition of the system, the amount of biogas generated and the amount of hydrogen produced using renewable energy for the methanation reaction may not be in balance, and excess carbon dioxide in the system may have to be released outside the system.

At least one embodiment of the present disclosure was made in view of the above circumstances, and an object thereof is to provide a biogas-utilizing methanation system that can reduce carbon dioxide emissions and enables clean power generation with high efficiency even when the supply amount of the to-be-treated gas or the power of the renewable energy power generation device fluctuates.

### Solution to the Problems

To solve the above problem, a biogas-utilizing methanation system according to an aspect of the present disclosure includes: a solid oxide fuel cell capable of generating power by using a to-be-treated gas containing methane and carbon dioxide as a fuel gas; a hydrogen production device capable of producing hydrogen by using power of a renewable energy power generation device; a methanation device capable of producing methane by methanation process using carbon dioxide contained in an exhaust fuel gas of the solid oxide fuel cell and the hydrogen produced by the hydrogen production device, and yielding the methane as a chemical raw material or supplying the methane to the solid oxide fuel cell as the fuel gas; a methane purification device capable of purifying a methane gas produced by the methanation device, and supplying at least part of the methane to outside as a chemical raw material and supplying an off-gas to the solid oxide fuel cell; and a storage device capable of storing at least part of the carbon dioxide supplied to the methanation device on the basis of at least one of supply amount of the to-be-treated gas or the power of the renewable energy power generation device.

To solve the above problem, a biogas-utilizing methanation system according to an aspect of the present disclosure includes: a hydrogen production device capable of producing hydrogen by using power of a renewable energy power generation device; a methanation device capable of producing methane by methanation process using a to-be-treated gas containing methane and carbon dioxide and the hydrogen produced by the hydrogen production device; a methane purification device capable of purifying the methane produced by the methanation device, and supplying at least part of the methane to outside as a chemical raw material and supplying an off-gas to a solid oxide fuel cell; the solid oxide fuel cell capable of generating power by using the off-gas of the methane purification device; and a storage device capable of storing at least part of the carbon dioxide supplied to the methanation device on the basis of at least one of supply amount of the to-be-treated gas or power of the renewable energy power generation device.

### Advantageous Effects

At least one embodiment of the present disclosure provides a biogas-utilizing methanation system that can reduce carbon dioxide gas emissions and enables clean power generation with high efficiency even when the supply amount of the to-be-treated gas or the power of the renewable energy power generation device fluctuates.

### BRIEF DESCRIPTION OF DRAWINGS

FIG. 1 is an overall configuration diagram of a biogas-utilizing methanation system according to an embodiment.

FIG. 2 is a diagram showing an operational pattern of the biogas-utilizing methanation system of FIG. 1 for each operational condition.

FIG. 3 is an overall configuration diagram of a biogas-utilizing methanation system according to another embodiment.

### DETAILED DESCRIPTION

Embodiments of the present invention will now be described in detail with reference to the accompanying drawings. It is intended, however, that unless particularly identified, dimensions, materials, shapes, relative positions, and the like of components described in the embodiments shall be interpreted as illustrative only and not intended to limit the scope of the present invention.

For instance, an expression of relative or absolute arrangement such as “in a direction”, “along a direction”, “parallel”, “orthogonal”, “centered”, “concentric” and “coaxial” shall not be construed as indicating only the arrangement in a strict literal sense, but also includes a state where the arrangement is relatively displaced by a tolerance, or by an angle or a distance whereby it is possible to achieve the same function.

For instance, an expression of an equal state such as “same” “equal” and “uniform” shall not be construed as indicating only the state in which the feature is strictly equal, but also includes a state in which there is a tolerance or a difference that can still achieve the same function.

Further, for instance, an expression of a shape such as a rectangular shape or a cylindrical shape shall not be construed as only the geometrically strict shape, but also includes a shape with unevenness or chamfered corners within the range in which the same effect can be achieved.

On the other hand, an expression such as “comprise”, “include”, “have”, “contain” and “constitute” are not intended to be exclusive of other components.

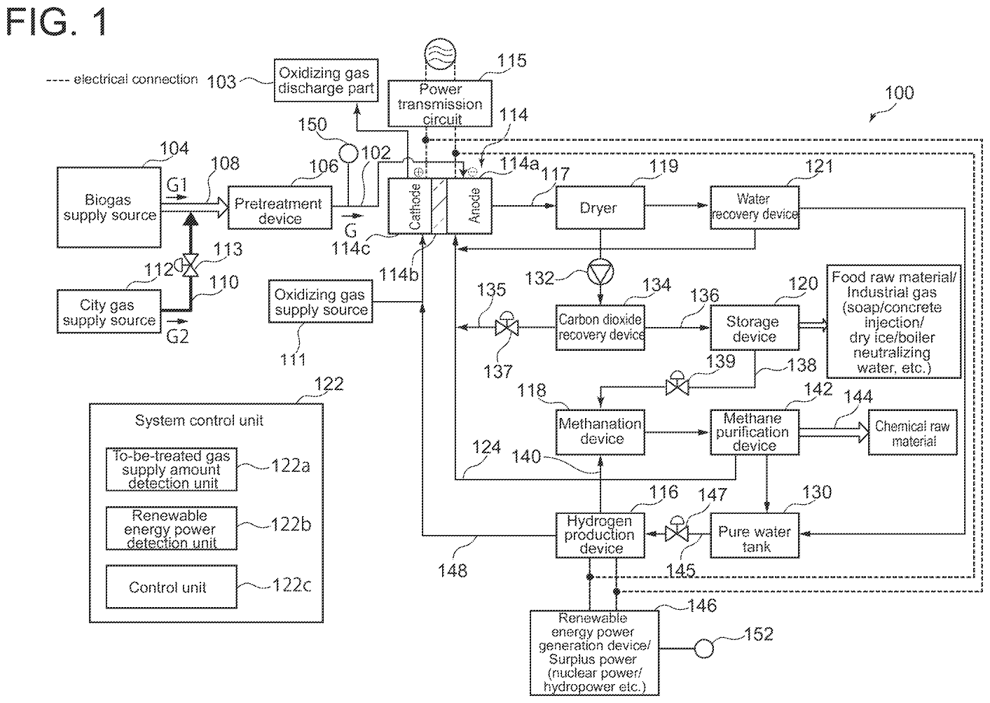

FIG. 1 is an overall configuration diagram of a biogas-utilizing methanation system 100 according to an embodiment. The biogas-utilizing methanation system 100 is a power generation system that can generate power using a to-be-treated gas G containing a combustible component as fuel. The to-be-treated gas G is, for example, biogas such as food residue gas discharged from a beer factory and containing methane as a combustible component. In the following embodiments, the case where biogas discharged from a beer factory is treated as the to-be-treated gas G will be described as an example, but the to-be-treated gas G may be, for example, digestion gas discharged from a sewage treatment facility, or boil off-gas generated in a tank for storing a liquefied natural gas (LNG).

The biogas-utilizing methanation system 100 is supplied with the to-be-treated gas G through a to-be-treated gas supply line 102. A biogas supply source 104 for supplying biogas G1 of a beer factory as a main component of the to-be-treated gas G and a pretreatment device 106 for performing pretreatment on the biogas G1 supplied from the biogas supply source 104 are disposed upstream of the to-be-treated gas supply line 102.

A city gas supply line 110 branches off from a biogas supply line 108 connecting the biogas supply source 104 and the pretreatment device 106, and a city gas supply source 112 is connected in parallel to the biogas supply source 104. The city gas supply source 112 can supply city gas G2 containing high purity methane. The city gas G2 supplied from the city gas supply source 112 can be mixed into the biogas G1 flowing through the biogas supply line 108 by controlling the opening degree of a city gas flow rate control valve 113 disposed on the city gas supply line 110. By adjusting the supply amount of the city gas G2 to the biogas G1 in this way, the supply amount of methane contained in the biogas G1 can be adjusted by the city gas G2.

A mixed gas of the biogas G1 and the city gas G2 is pretreated by the pretreatment device 106. The pretreatment is a process for refining the mixed gas into the to-be-treated gas G suitable for the biogas-utilizing methanation system 100. For example, the pretreatment device 106 refines the to-be-treated gas G by performing desulfurization treatment on the mixed gas to desulfurize sulfur in the mixed gas.

The biogas-utilizing methanation system 100 includes a solid oxide fuel cell 114, a hydrogen production device 116, a methanation device 118, a storage device 120, and a system control unit 122.

The solid oxide fuel cell 114 is a power generation device configured to generate power by chemical reaction between fuel gas and oxidizing gas, and has characteristics such as excellent power generation efficiency and environmental friendliness. The solid oxide fuel cell 114 includes an anode 114a, an electrolyte 114b, and a cathode 114c.

The anode 114a is composed of a composite of Ni and zirconia-based electrolyte material, for example, Ni/YSZ. In this case, in the anode 114a, Ni, which is a component of the anode 114a, has catalysis on the to-be-treated gas G. By this catalysis, methane contained in the to-be-treated gas G supplied to the solid oxide fuel cell 114 reacts with water vapor recovered from a methane purification device and an exhaust fuel gas and is reformed into hydrogen (H₂) and carbon monoxide (CO). Further, the anode 114a causes hydrogen (H₂) and carbon monoxide (CO) obtained by reforming to electrochemically react with oxygen ions (O²⁻) supplied from the cathode 114c via the electrolyte 114b in the vicinity of the interface with the electrolyte 114b to produce water (H₂O) and carbon dioxide (CO₂). The exhaust fuel gas from the anode 114a after such reaction is discharged through a first exhaust fuel gas line 117.

For the electrolyte 114b, ceramic such as zirconia ceramic is used. The electrolyte 114b moves oxygen ions (O²—) generated in the cathode 114c to the anode 114a.

The cathode 114c is composed of, for example, a LaSrMnO₃-based oxide or a LaCoO₃-based oxide. The cathode 114c reduces oxygen in a supplied oxidizing gas such as air in the vicinity of the interface with the electrolyte 114b to generate oxygen ions (O²⁻). The remaining exhaust oxidizing gas after oxygen ions have been supplied to the electrolyte 114b in the cathode 114c can be discharged to the outside from an oxidizing gas discharge part 103.

The solid oxide fuel cell 114 may be configured to independently discharge the exhaust fuel gas from the anode 114a and the exhaust oxidizing gas from the cathode 114c to the outside. Specifically, the exhaust fuel gas from the anode 114a can be taken out from the first exhaust fuel gas line 117, while the exhaust oxidizing gas from the cathode 114c can be taken out from the oxidizing gas discharge part 103. In the present embodiment, as described above, oxygen in the oxidizing gas at the cathode 114c moves to the anode 114a as oxygen ions through the electrolyte 114b of the solid oxide fuel cell, and reacts at the anode 114a with methane in the to-be-treated gas G and carbon monoxide generated by the reforming reaction to produce carbon dioxide in the exhaust fuel gas, so in principle the concentration of carbon dioxide in the exhaust fuel gas is higher than that in the exhaust gas from a normal combustion facility. On the other hand, in a so-called non-sealed solid oxide fuel cell where the exhaust fuel gas and the exhaust oxidizing gas are combusted within the stack, nitrogen in the oxidizing gas is mixed into the exhaust fuel gas, so that the concentration of carbon dioxide is diluted. Exhaust gas from non-sealed solid oxide fuel cells and gas engines contains a few percent carbon dioxide and about 80% nitrogen, while exhaust gas from the present sealed solid oxide fuel cell contains high percentage, namely 35 to 45% of carbon dioxide. In the present embodiment, since the concentration of carbon dioxide in the exhaust fuel gas from the anode 114a is high, the power required to recover carbon dioxide can be reduced, and as will be described later, carbon dioxide contained in the exhaust fuel gas can be effectively used.

The anode 114a of the solid oxide fuel cell 114 is supplied with, as the fuel gas, at least one of the to-be-treated gas G or methane produced by the methanation device 118. The ratio of the to-be-treated gas G and methane produced by the methanation device 118 in the fuel gas is variable depending on the operational pattern, as described later. The cathode 114c of the solid oxide fuel cell 114 is supplied with, as the oxidizing gas for reacting with the fuel gas, both or at least one of oxidizing gas (air) from an oxidant supply source 111 or oxygen which is by-product when hydrogen is produced by the hydrogen production device 116 through an oxidant supply line 148.

The solid oxide fuel cell 114 generates power through reaction between the fuel gas and the oxidizing gas. The power generated by the solid oxide fuel cell 114 can be supplied to an external power system (e.g., in-house power system or commercial power system) via a power transmission circuit 115 from the output terminal (shown by the dotted line) of the solid oxide fuel cell 114 according to power demand.

The detailed structure of the solid oxide fuel cell 114 is in accordance with known examples and will not be described herein.

The exhaust fuel gas discharged from the anode 114a of the solid oxide fuel cell 114 contains carbon dioxide and water which are products of the power generation reaction, and methane, carbon monoxide, and hydrogen which have not been consumed in the power generation reaction. The exhaust fuel gas discharged from the anode 114a is introduced through the first exhaust fuel gas line 117 to a dryer 119, where moisture contained in the exhaust fuel gas is removed. The moisture removed by the dryer 119 is recovered by a water recovery device 121, and is used as steam for reforming methane flowing through a produced methane supply line 124, which will be described later, and is also stored in a pure water tank 130.

The exhaust oxidizing gas discharged from the cathode 114c does not contain carbon dioxide and can be discharged to the outside from the oxidizing gas discharge part 103 as clean gas.

The exhaust fuel gas from which moisture has been removed by the dryer 119 is introduced to a carbon dioxide recovery device 134 by a recycle gas compressor 132. Various carbon dioxide recovery methods are available, including chemical absorption method using an absorption agent (such as amine absorption liquid), physical absorption method (such as PSA and TSA) using an absorption agent, membrane separation method, and cryogenic distillation method. The appropriate method is selected based on conditions such as throughput, carbon dioxide concentration in the exhaust fuel gas, supply pressure, and temperature. The carbon dioxide recovery device 134 can at least partially recover carbon dioxide contained in the exhaust fuel gas, and the recovery amount is variable depending on the operational pattern described later.

Carbon dioxide recovered by the carbon dioxide recovery device 134 is stored into the storage device 120 through a carbon dioxide storage line 136. The storage device 120 is, for example, a tank facility capable of storing carbon dioxide, and has a capacity capable of storing the maximum amount of carbon dioxide when it becomes excessive in the system. Therefore, even when carbon dioxide becomes excessive in the system, the excess carbon dioxide can be stored in the storage device 120 and is not emitted to the outside. Further, the exhaust fuel gas from which carbon dioxide has been recovered by the carbon dioxide recovery device 134 is returned to the solid oxide fuel cell 114 through a second exhaust fuel gas line 135.

Carbon dioxide stored in the storage device 120 can be appropriately taken out as an industrial gas for food raw material, soap, concrete injection, dry ice, boiler neutralizing water, etc.

The storage device 120 is connected to the methanation device 118 via a storage gas supply line 138. The storage gas supply line 138 is provided with a storage gas supply amount control valve 139. By controlling the opening degree of the storage gas supply amount control valve 139, carbon dioxide stored in the storage device 120 can be supplied to the methanation device 118 in an amount necessary for the methanation reaction.

In the methanation device 118, methanation is performed through reaction between carbon dioxide introduced through the storage gas supply line 138 and high-purity hydrogen supplied from the hydrogen production device 116 through a hydrogen supply line 140 to produce methane from carbon dioxide and hydrogen. For the methanation process, a direct method or an indirect method as represented by the following chemical reaction formulae may be used.

### (Direct Method)

CO₂+4H₂→CH₄+2H₂O−39.4 kcal/mol

### (Indirect Method)

CO₂+H₂→CO+H₂O+9.8 kcal/mol

CO+3H₂→CH₄+H₂O−49.3 kcal/mol

The methane produced by the methanation device 118 is introduced to a methane purification device 142. The methane purification device 142 purifies the methane produced by the methanation device 118 to produce high-purity methane. The methane purified by the methane purification device 142 can be taken out as a chemical raw material through a methane discharge line 144. Further, the remaining off-gas after purification may be supplied as the fuel gas to the solid oxide fuel cell 114 through a methane supply line 124. The ratio of methane supplied to the solid oxide fuel cell 114 through the produced methane supply line 124 and methane taken out through the methane discharge line 144 is variable depending on the operational pattern as described later.

The hydrogen production device 116 is a device capable of generating hydrogen using the power of a renewable energy power generation device or a surplus power 146. Specifically, it produces hydrogen by electrolyzing pure water stored in the pure water tank 130 using the power of the renewable energy power generation device or the surplus power 146. The renewable energy power generation device can generate power using renewable energy in a manner that does not emit carbon dioxide, and the surplus power can generate power in a manner that does not emit carbon dioxide, such as nuclear power generation or hydropower generation. Using the power thus generated for the hydrogen production device 116 is effective in reducing carbon dioxide emissions in the system. Further, since the power generation amount of the renewable energy power generation device varies with the seasons or times in a day, for example, if it is difficult to cover the power required by the hydrogen production device 116 with only the power generation amount of the renewable energy power generation device, the shortage can be covered by using the surplus power.

The hydrogen production device 116 may supply hydrogen produced by the water electrolysis to the methanation device 118 through the hydrogen supply line 140, and supply oxygen produced as by-product of the water electrolysis to the solid oxide fuel cell 114 as the oxidizing gas through the oxidant supply line 148. Thus, by increasing the concentration of oxygen contained in the oxidizing gas, the power generation performance of the solid oxide fuel cell 114 is improved.

The pure water tank 130 is connected to the hydrogen production device 116 via a pure water supply line 145. The pure water supply line 145 is provided with a pure water supply amount control valve 147, and the amount of pure water supplied to the hydrogen production device 116 can be adjusted by controlling the opening degree of the pure water supply amount control valve 147.

The system control unit 122 is, for example, a control unit for controlling the above-described elements constituting the biogas-utilizing methanation system 100. The system control unit 122 includes, for example, a central processing unit (CPU), a random access memory (RAM), a read only memory (ROM), and a storage medium or the like that is readable with a computer. Then, a series of processes for realizing the various functions is stored in the storage medium or the like in the form of a program, as an example. The CPU reads the program out to the RAM or the like and executes processing/calculation of information, thereby realizing the various functions. The program may be applied with a configuration where the program is installed in the ROM or another storage medium in advance, a configuration where the program is provided in a state of being stored in the computer-readable storage medium, a configuration where the program is distributed via a wired or wireless communication means, or the like. The computer-readable storage medium is a magnetic disk, a magneto-optical disk, a CD-ROM, a DVD-ROM, a semiconductor memory, or the like.

The system control unit 122 includes a to-be-treated gas supply amount detection unit 122a for detecting the supply amount Qs of the to-be-treated gas supplied to the biogas-utilizing methanation system 100, a renewable energy power detection unit 122b for detecting the power Pa of the renewable energy power generation device 146, and a control unit 122c for controlling the components of the biogas-utilizing methanation system 100 on the basis of operational conditions specified from detection results of the to-be-treated gas supply amount detection unit 122a and the renewable energy power detection unit 122b.

The to-be-treated gas supply amount detection unit 122a detects the supply amount Qs of the to-be-treated gas G to the biogas-utilizing methanation system 100 by acquiring a detection value of a supply amount sensor 150 disposed in the to-be-treated gas supply line 102. Further, the to-be-treated gas supply amount detection unit 122a may use the result of estimating the supply amount Qs of the to-be-treated gas G from the supply amount of the biogas G1 from the biogas supply source 104 in a feedforward manner, on the basis of data on the operation plan of the beer factory (biogas supply source 104), which is the supply source of the biogas G1, and data on environmental factors such as weather information which affect the production amount of the beer factory, instead of or in addition to the measurement result of a measuring device such as the supply amount sensor 150, for example.

The renewable energy power detection unit 122b detects the power Pa of the renewable energy power generation device 146. The power Pa is obtained as an average amount of power detected by a power sensor 152 disposed in the renewable energy power generation device 146 for a certain time, for example.

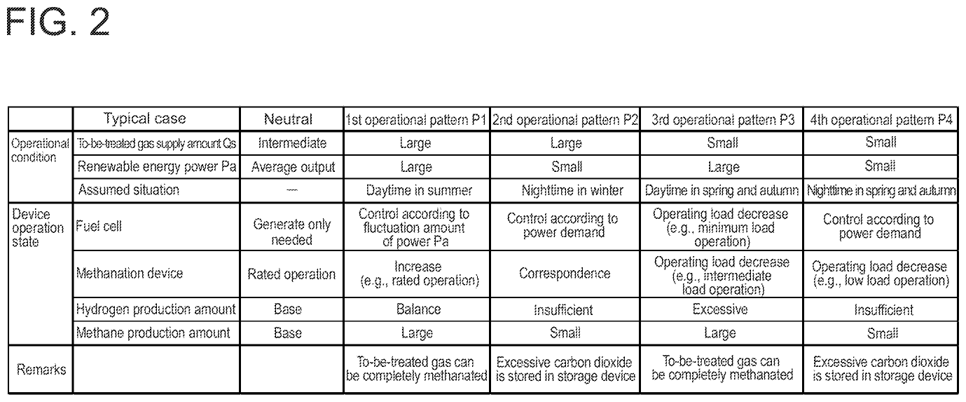

The control of the biogas-utilizing methanation system 100 by the system control unit 122 is classified into some operational patterns based on operational conditions specified from detection results of the to-be-treated gas supply amount detection unit 122a and the renewable energy power detection unit 122b. FIG. 2 is a diagram showing an operational pattern of the biogas-utilizing methanation system 100 of FIG. 1 for each operational condition. In this embodiment, the operational patterns of the biogas-utilizing methanation system 100 include a first operational pattern P1 to a fourth operational pattern P4.

The first operational pattern P1 is an operational pattern corresponding to an operational condition in which the supply amount Qs of the to-be-treated gas G is large and the power Pa of the renewable energy power generation device 146 is large relative to the supply amount Qs of the to-be-treated gas G. That is, the first operational pattern P1 is selected when the supply amount Qs of the to-be-treated gas G increases and the power Pa of the renewable energy power generation device 146 increases relative to a neutral operational condition (e.g., when the supply amount Qs of the to-be-treated gas G and the power Pa of the renewable energy power generation device 146 are annual average values). Such an operational condition corresponds to, for example, “daytime in summer” where the emission amount of the to-be-treated gas G increases with the increase in the beer production in beer factories, and the renewable energy such as solar energy increases with the increase in sunlight hours.

In the first operational pattern P1, the control unit 122c controls the solid oxide fuel cell 114 so as to respond to the fluctuation amount of the power Pa, and also controls the methanation device 118 to increase the operating load. In this case, both the supply amount Qs of the to-be-treated gas G and the power Pa of the renewable energy power generation device 146 are abundant. Therefore, the solid oxide fuel cell 114 can generate power to meet relatively large power demand and to absorb fluctuations in the power Pa of the renewable energy power generation device 146. Further, by increasing the operating load of the methanation device 118, carbon dioxide contained in the to-be-treated gas, which is increasing in supply, and carbon dioxide stored in the storage device 120 are methanated to increase the production of methane. In this case, carbon dioxide in the storage device 120 is methanated and supplied to the outside as methane, so carbon dioxide generated even when the power Pa decreases can be temporarily stored in the storage device 120 and is not discharged to the outside.

The second operational pattern P2 is an operational pattern corresponding to an operational condition in which the supply amount Qs of the to-be-treated gas G is large and the power Pa of the renewable energy power generation device 146 is small. That is, the second operational pattern P2 is selected when the supply amount Qs of the to-be-treated gas G increases and the power Pa of the renewable energy power generation device 146 decreases relative to a neutral operational condition (e.g., when the supply amount Qs of the to-be-treated gas G and the power Pa of the renewable energy power generation device 146 are annual average values). Such an operational condition corresponds to, for example, “nighttime in winter” where the emission amount of the to-be-treated gas G increases with the increase in the beer production in beer factories, but the renewable energy such as solar energy decreases with the decrease in sunlight hours.

In the second operational pattern P2, the control unit 122c controls the power generation amount of the solid oxide fuel cell 114 according to power demand, and controls the storage device 120 to store excess carbon dioxide relative to the amount of hydrogen produced by the hydrogen production device 116. In this case, since the power Pa of the renewable energy power generation device 146 is insufficient relative to the supply amount Qs of the to-be-treated gas G, the amount of hydrogen produced by the hydrogen production device 116 is less than the amount required for the methanation device 118 to process all the carbon dioxide in the system. Therefore, by storing excess carbon dioxide in the system (i.e., carbon dioxide that cannot be methanated) temporarily in the storage device 120, it is possible to prevent carbon dioxide emissions to the outside. On the other hand, the solid oxide fuel cell 114 generates the minimum necessary power within the range according to power demand to reduce the exhaust fuel gas containing carbon dioxide and suppress the amount of carbon dioxide that becomes excessive in the system.

Further, in the second operational pattern P2, since the power generation amount of the renewable energy power generation device is small, the surplus power is used instead of or in addition to the renewable energy device to produce hydrogen in the hydrogen production device 116 required for methanation of carbon dioxide stored in the storage device 120. Thus, even in a situation where the power generation amount of the renewable energy device is small, since the surplus power can be used to process carbon dioxide, the amount of carbon dioxide stored in the storage device 120 does not become excessive. In other words, since the storage capacity of carbon dioxide required for the storage device 120 can be reduced, the storage device 120 can be made compact.

The carbon dioxide temporarily stored in the storage device 120 in the second operational pattern P2 may be methanated by the methanation device 118 when the hydrogen production amount of the hydrogen production device 116 increases with the recovery of the power Pa of the renewable energy power generation device 146 to produce methane without emitting carbon dioxide gas to the outside. Further, the recovered carbon dioxide can be taken out and used for an industrial gas such as food raw material, soap, concrete injection, dry ice, boiler neutralizing water, etc., without being emitted into the atmosphere.

The third operational pattern P3 is an operational pattern corresponding to an operational condition in which the supply amount Qs of the to-be-treated gas G is small and the power Pa of the renewable energy power generation device 146 is large. That is, the third operational pattern P3 is selected when the supply amount Qs of the to-be-treated gas G decreases and the power Pa of the renewable energy power generation device 146 increases relative to a neutral operational condition (e.g., when the supply amount Qs of the to-be-treated gas G and the power Pa of the renewable energy power generation device 146 are annual average values). Such an operational condition corresponds to, for example, “daytime in spring and autumn” where the emission amount of the to-be-treated gas G decreases with the decrease in the beer production in beer factories, and the renewable energy such as solar energy increases with the increase in sunlight hours.

In the third operational pattern P3, the control unit 122c controls the solid oxide fuel cell 114 and the methanation device 118 to decrease the operating loads. In this case, since the power Pa of the renewable energy power generation device 146 is sufficiently large relative to the supply amount Qs of the to-be-treated gas G, the hydrogen production device 116 can supply hydrogen required to methanate all the carbon dioxide in the system. Therefore, all the carbon dioxide contained in the to-be-treated gas G and in the exhaust fuel gas from the solid oxide fuel cell 114 operated at low operating load can be methanated by the methanation device 118, so that no carbon dioxide is emitted to the outside.

The fourth operational pattern P4 is an operational pattern corresponding to an operational condition in which the supply amount Qs of the to-be-treated gas G is small and the power Pa of the renewable energy power generation device 146 is small. That is, the fourth operational pattern P4 is selected when the supply amount Qs of the to-be-treated gas G decreases and the power Pa of the renewable energy power generation device 146 decreases relative to a neutral operational condition (e.g., when the supply amount Qs of the to-be-treated gas G and the power Pa of the renewable energy power generation device 146 are annual average values). Such an operational condition corresponds to, for example, “nighttime in spring and autumn” where the emission amount of the to-be-treated gas G decreases with the decrease in the beer production in beer factories, and the renewable energy such as solar energy decreases with the decrease in sunlight hours.

In the fourth operational pattern P4, the control unit 122c controls the power generation amount of the solid oxide fuel cell 114 according to power demand, and controls the storage device 120 to temporarily store excess carbon dioxide relative to the amount of hydrogen produced by the hydrogen production device 116. In this case, the solid oxide fuel cell 114 discharges the exhaust fuel gas according to power demand, but since the power Pa of the renewable energy power generation device 146 is small, the hydrogen production device 116 cannot provide sufficient hydrogen to methanate all the carbon dioxide in the system, resulting in excess carbon dioxide in the system. By temporarily storing such excess carbon dioxide in the system in the storage device 120, it is possible to prevent carbon dioxide emissions to the outside.

The carbon dioxide stored in the storage device 120 in the fourth operational pattern P4 may be methanated by the methanation device 118 when the hydrogen production amount of the hydrogen production device 116 increases with the recovery of the power Pa of the renewable energy power generation device 146 or the production amount of the hydrogen production device 116 increases by the surplus power to produce methane without emitting carbon dioxide gas to the outside. Further, the recovered carbon dioxide can be taken out and used for an industrial gas such as food raw material, soap, concrete injection, dry ice, boiler neutralizing water, etc., without being emitted into the atmosphere.

As described above, the biogas-utilizing methanation system 100 is controlled by operational patterns based on operational conditions specified by the supply amount Qs of the to-be-treated gas G and the power Pa of the renewable energy power generation device 146. This enables methane production without carbon dioxide gas emissions and clean power generation with high efficiency even when the supply amount Qs of the to-be-treated gas G or the power Pa of the renewable energy power generation device 146 fluctuates.

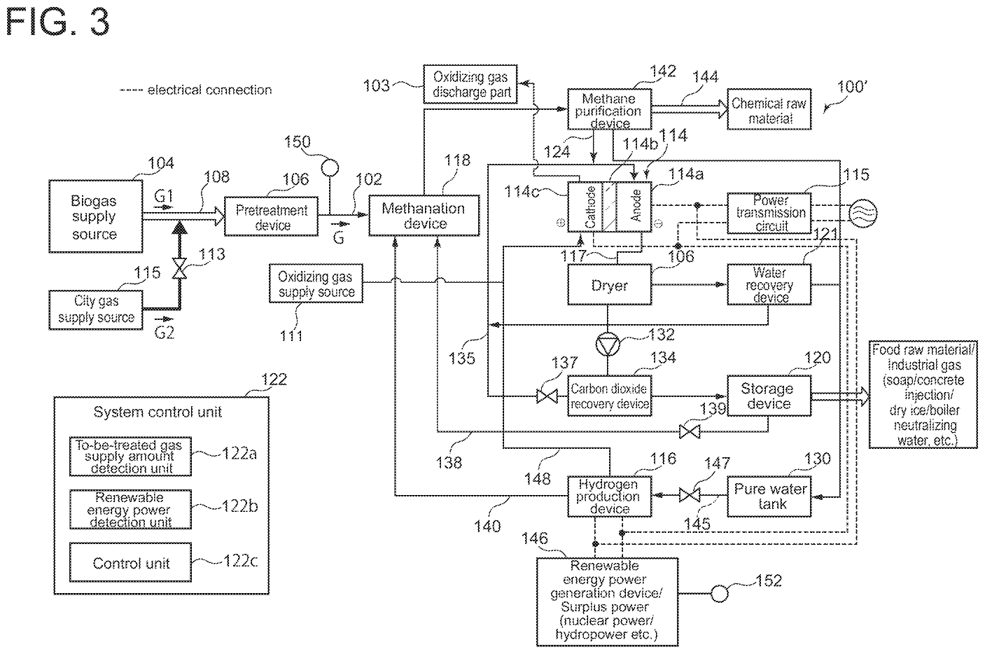

Next, another embodiment of the biogas-utilizing methanation system 100 will be described. FIG. 3 is an overall configuration diagram of a biogas-utilizing methanation system 100′ according to another embodiment. The biogas-utilizing methanation system 100′ shares the main configuration with the biogas-utilizing methanation system 100 described above, but differs at least partially in the layout of components within the system. In the following description, the same features as those in biogas-utilizing methanation system 100 are associated with the same reference numerals, and not described again.

The methanation device 118 of the biogas-utilizing methanation system 100′ is supplied with the to-be-treated gas G through a to-be-treated gas supply line 102. The to-be-treated gas G is biogas G1 discharged from a beer factory, as described above, and contains methane and carbon dioxide. Further, carbon dioxide is introduced to the methanation device 118 through a storage gas line 138. The carbon dioxide introduced to the methanation device 118 reacts with hydrogen supplied from the hydrogen production device 116 through a hydrogen supply line 140 for methanation to produce methane.

The methane produced by the methanation device 118 is introduced to a methane purification device 142 and thereby purified. At least part of the methane produced by the methane purification device 142 can be supplied to the outside as a chemical raw material, and the off-gas can be supplied to the solid oxide fuel cell 114 through a produced methane supply line 124. The solid oxide fuel cell 114 is supplied with, as the fuel gas, exhaust fuel gas which is discharged from the anode 114a of the solid oxide fuel cell 114 and from which moisture and carbon dioxide have been at least partially recovered, or the off-gas which mainly contains methane from the methane purification device 142 through the produced methane supply line 124. The cathode 114c of the solid oxide fuel cell 114 is supplied with, as the oxidizing gas for reacting with the fuel gas, both or at least one of oxidizing gas (air) from an oxidant supply source 111 or oxygen which is by-product when hydrogen is produced by the hydrogen production device 116 through an oxidant supply line 148.

The solid oxide fuel cell 114 generates power through reaction between the fuel gas and the oxidizing gas. The power generated by the solid oxide fuel cell 114 can be supplied to an external power system (e.g., commercial power system) via a power transmission circuit 115 from the output terminal (shown by the dotted line) of the solid oxide fuel cell 114 according to power demand.

The methane purification device 142 purifies methane produced by the methanation device 118 to produce high-purity methane. The methane purified by the methane purification device 142 can be taken out as a chemical raw material through a methane discharge line 144. Further, the remaining off-gas after purification may be supplied as the fuel gas to the anode 114a of the solid oxide fuel cell 114 through a produced methane supply line 124.

The exhaust fuel gas discharged from the anode 114a of the solid oxide fuel cell 114 is introduced through a first exhaust fuel gas line 117 to a dryer 119, where moisture contained in the exhaust fuel gas is removed. The moisture removed by the dryer 119 is recovered by a water recovery device 121, and is partially used as steam for reforming methane flowing through a second exhaust fuel gas line 135, and is also stored in a pure water tank 130 and supplied to a hydrogen production device.

The exhaust fuel gas from which moisture has been removed by the dryer 119 is introduced to a carbon dioxide recovery device 134 by a recycle gas compressor 132. Various carbon dioxide recovery methods are available, including chemical absorption method using an absorption agent (such as amine absorption liquid), physical absorption method (such as PSA and TSA) using an absorption agent, membrane separation method, and cryogenic distillation method. The appropriate method is selected based on conditions such as throughput, carbon dioxide concentration in the exhaust fuel gas, supply pressure, and temperature. The carbon dioxide recovery device 134 can at least partially recover carbon dioxide contained in the exhaust fuel gas, and the recovery amount is variable depending on the operational pattern described above. Carbon dioxide recovered by the carbon dioxide recovery device 134 is stored into a carbon dioxide storage device through a carbon dioxide storage line 136. At least part of the exhaust fuel gas from which carbon dioxide has been recovered is supplied to the anode 114a of the solid oxide fuel cell 114 through the second exhaust fuel gas line 135 and reused.

In the biogas-utilizing methanation system 100′ having the above configuration, similarly, the control unit 122c of the system control unit 122 controls the components of the biogas-utilizing methanation system 100′ on the basis of operational conditions specified from detection results of the to-be-treated gas supply amount detection unit 122a and the renewable energy power detection unit 122b. The biogas-utilizing methanation system 100′ differs from the biogas-utilizing methanation system 100 (see FIG. 1) at least partially in the layout of the components, but can similarly control the four operational patterns, described above with reference to FIG. 2, based on the operational conditions. Thus, the biogas-utilizing methanation system 100′ also enables clean power generation with high efficiency without carbon dioxide gas emissions even when the supply amount Qs of the to-be-treated gas G or the power Pa of the renewable energy power generation device 146 fluctuates.

In addition, the components in the above-described embodiments may be appropriately replaced with known components without departing from the spirit of the present disclosure, or the above-described embodiments may be appropriately combined.

The contents described in the above embodiments would be understood as follows, for instance.

(1) A biogas-utilizing methanation system according to an aspect includes: a solid oxide fuel cell capable of generating power by using a to-be-treated gas containing methane and carbon dioxide as a fuel gas; a hydrogen production device capable of producing hydrogen by using power of a renewable energy power generation device; a methanation device capable of producing methane by methanation process using carbon dioxide contained in an exhaust fuel gas of the solid oxide fuel cell and the hydrogen produced by the hydrogen production device, and yielding the methane as a chemical raw material or supplying the methane to the solid oxide fuel cell as the fuel gas; a methane purification device capable of purifying a methane gas produced by the methanation device, and supplying at least part of the methane to outside as a chemical raw material and supplying an off-gas to the solid oxide fuel cell; and a storage device capable of storing at least part of the carbon dioxide supplied to the methanation device on the basis of at least one of supply amount of the to-be-treated gas or the power of the renewable energy power generation device.

According to the above aspect (1), the to-be-treated gas containing methane and carbon dioxide is used as the fuel gas of the solid oxide fuel cell, carbon dioxide in the exhaust fuel gas is recovered and used in the methanation device for reaction with hydrogen gas produced by using the power of the renewable energy power generation device to produce methane, and the methane is provided to the outside as a chemical raw material while the off-gas of the methane purification device is reused as the fuel gas of the solid oxide fuel cell, so that carbon dioxide emissions to the outside are reduced.

Further, although the power of the renewable energy power generation device varies depending on the sunshine conditions and weather conditions, the influence of power variations is absorbed by adjusting the operational balance of the solid oxide fuel cell, the hydrogen production device, and the methanation device. Thus, it is possible to respond to variations in production of biogas and power of renewable energy without using power storage equipment such as a large-capacity battery, which is disadvantageous in terms of cost.

Further, when carbon dioxide becomes excessive in the system relative to the processing capacity of the methanation device, the carbon dioxide can be stored in the storage device. Thus, when there is a margin in the processing capacity of the methanation device, the carbon dioxide stored in the storage device can be subject to methanation, and when there is a temporary excess of carbon dioxide in the system, the carbon dioxide can be stored in the storage device in a buffering manner, so that a clean biogas methanation system that does not emit carbon dioxide to the outside can be achieved. In particular, since the required number of moles of carbon dioxide is smaller than that of hydrogen in the methanation reaction, by selecting carbon dioxide as the object to be stored in the storage device, the capacity of the storage device can be reduced, and the above-described system can be constructed with a compact configuration.

(2) A biogas-utilizing methanation system according to an aspect includes: a hydrogen production device capable of producing hydrogen by using power of a renewable energy power generation device; a methanation device capable of producing methane by methanation process using a to-be-treated gas containing methane and carbon dioxide and the hydrogen produced by the hydrogen production device; a methane purification device capable of purifying the methane produced by the methanation device, and supplying at least part of the methane to outside as a chemical raw material and supplying an off-gas to a solid oxide fuel cell; the solid oxide fuel cell capable of generating power by using the off-gas of the methane purification device; and a storage device capable of storing at least part of the carbon dioxide supplied to the methanation device on the basis of at least one of supply amount of the to-be-treated gas or power of the renewable energy power generation device.

According to the above aspect (2), by methanating carbon dioxide gas contained in the to-be-treated gas with hydrogen gas generated using the power of the renewable energy power generation device to produce methane gas, it is possible to make use of the to-be-treated gas. The methane gas thus produced can be supplied to the outside as a chemical raw material, and the purified off-gas can be supplied to the solid oxide fuel cell as the fuel gas.

Further, although the power of the renewable energy power generation device varies depending on the sunshine conditions and weather conditions, the influence of power variations is absorbed by adjusting the operational balance of the solid oxide fuel cell, the hydrogen production device, and the methanation device. Thus, it is possible to respond to variations in power of renewable energy without using power storage equipment such as a large-capacity battery, which is disadvantageous in terms of cost.

Further, when carbon dioxide becomes excessive in the system relative to the processing capacity of the methanation device, the carbon dioxide can be stored in the storage device. Thus, when there is a margin in the processing capacity of the methanation device, the carbon dioxide stored in the storage device can be subject to methanation, and when there is a temporary excess of carbon dioxide in the system, the carbon dioxide can be stored in the storage device in a buffering manner, so that a clean biogas methanation system that does not emit carbon dioxide to the outside can be achieved. In particular, since the required number of moles of carbon dioxide is smaller than that of hydrogen in the methanation reaction, by selecting carbon dioxide as the object to be stored in the storage device, the capacity of the storage device can be reduced, and the above-described system can be constructed with a compact configuration.

(3) In another aspect, in the above aspect (1) or (2), the storage device is capable of storing carbon dioxide recovered from an exhaust fuel gas of the solid oxide fuel cell, is connected to the methanation device via a storage gas supply line provided with a flow rate control valve, and is capable of supplying carbon dioxide to outside as a food raw material or an industrial gas.

According to the above aspect (3), the storage device is connected to the methanation device via the storage gas supply line. The storage gas supply line is provided with the flow rate control valve. By controlling the opening degree of the flow rate control valve, carbon dioxide stored in the storage device can be supplied to the methanation device at a freely-selected timing. Thus, by supplying carbon dioxide stored in the storage device to the methanation device through the storage gas supply line at a timing when the methanation device has sufficient processing capacity, i.e., hydrogen supply capacity, carbon dioxide that temporarily becomes excessive in the system can be converted into methane without being emitted to the outside, and the carbon dioxide can be used as a food raw material or an industrial gas.

(4) In another aspect, in any one of the above aspects (1) to (3), the biogas-utilizing methanation system is configured to supply the power generated by the solid oxide fuel cell and surplus power to the hydrogen production device when the power of the renewable energy power generation device is insufficient.

According to the above aspect (4), when the power required to produce hydrogen by the hydrogen production device is insufficient due to fluctuations in the power of the renewable energy power generation device, the power generated by the solid oxide fuel cell can be supplied to compensate for the shortage, and when the power of the renewable energy power generation device is insufficient for a long period of time, the surplus power such as nuclear power and hydropower can be received to cover the power required to produce hydrogen.

(5) In another aspect, in any of the above (1) to (4), the biogas-utilizing methanation system is configured to supply oxygen produced by the hydrogen production device as part of an oxidizing gas of the solid oxide fuel cell.

According to the above aspect (5), oxygen produced when the hydrogen production device produces hydrogen can be used as part of the oxidizing gas supplied to the solid oxide fuel cell.

(6) In another aspect, in any one of the above aspects (1) to (5), the biogas-utilizing methanation system further includes a methane supply source capable of supplying methane to the to-be-treated gas.

According to the above aspect (6), by supplying methane to the to-be-treated gas from the methane supply source, the methane supply amount in the to-be-treated gas can be adjusted, and the system can be stably operated according to power demand.

(7) In another aspect, in any one of the above aspects (1) to (6), the solid oxide fuel cell is capable of discharging an exhaust fuel and an exhaust oxidizing gas independently.

In the present disclosure, the above-described system can be achieved by using the solid oxide fuel cell capable of independently extracting the exhaust fuel gas and the exhaust oxidizing gas as the fuel cell. In this case, oxygen in the oxidizing gas moves to the fuel side as oxygen ions through the electrolyte of the solid oxide fuel cell, and reacts with methane and carbon monoxide in the fuel to produce carbon dioxide in the exhaust fuel, so in principle the concentration of carbon dioxide is higher than that in the exhaust gas from a normal combustion facility. On the other hand, in a non-sealed solid oxide fuel cell where the exhaust fuel gas and the exhaust oxidizing gas are combusted within the stack, the exhaust fuel gas is diluted with nitrogen in the air, so that the concentration of carbon dioxide is reduced to about 1/10.

Further, in the case of using another device such as a polymer electrolyte fuel cell (PEFC) or a phosphoric acid fuel cell (PAFC) as the fuel cell, it is necessary to reform methane into hydrogen in advance, and at this time, carbon dioxide is generated from a heating burner of the reformer, and thus the benefits of the present system cannot be enjoyed. Further, in the case of using a device such as a gas engine, it is necessary to perform oxygen combustion, which requires the use of oxygen generated by the water electrolysis device or an additional oxygen production device, resulting in an inevitable reduction in the operability and energy efficiency of the system.

(8) In another aspect, in any one of the above aspects (1) to (7), the biogas-utilizing methanation system further includes a system control unit for controlling at least part of the solid oxide fuel cell, the hydrogen production device, the methanation device, or the storage device, on the basis of at least one of the supply amount of the to-be-treated gas or the power of the renewable energy power generation device.

According to the above aspect (8), the components of the biogas-utilizing methanation system are controlled on the basis of at least one of the supply amount of the to-be-treated gas or the power of the renewable energy power generation device. Thus, even when the supply amount of the to-be-treated gas or the power of the renewable energy power generation device fluctuates, the biogas-utilizing methanation system enables clean operation with reduced carbon dioxide emissions by adjusting the operational balance.

(9) In another aspect, in the above aspect (8), the system control unit is configured to, when the supply amount of the to-be-treated gas increases and the power of the renewable energy power generation device increases, control the solid oxide fuel cell according to fluctuation amount of the power, and control the methanation device to increase an operating load.

According to the above aspect (9), when both the supply amount of the to-be-treated gas and the power of the renewable energy power generation device are abundant, the solid oxide fuel cell generates power to meet relatively large power demand and to absorb fluctuations in the power of the renewable energy power generation device. Further, by increasing the operating load of the methanation device, carbon dioxide contained in the to-be-treated gas, which is increasing in supply, and carbon dioxide contained in the exhaust fuel gas of the solid oxide fuel cell can be completely methanated, so that the carbon dioxide emissions to the outside can be prevented. Additionally, increasing the production of methane and supplying it as a chemical raw material improves the economic benefits of the system operation.

(10) In another aspect, in the above aspect (8) or (9), the system control unit is configured to, when the supply amount of the to-be-treated gas increases and the power of the renewable energy power generation device decreases, control power generation amount of the solid oxide fuel cell according to power demand, control the storage device to store excess carbon dioxide relative to production amount of the hydrogen produced by the hydrogen production device, and use surplus power to ensure that storage amount of carbon dioxide is not excessive.

According to the above aspect (10), when the power of the renewable energy power generation device is insufficient relative to the supply amount of the to-be-treated gas, the amount of hydrogen produced by the hydrogen production device is less than the amount required for the methanation device to process all the carbon dioxide in the system. Therefore, by storing excess carbon dioxide in the system (i.e., carbon dioxide that cannot be methanated) temporarily in the storage device, it is possible to prevent carbon dioxide emissions to the outside. On the other hand, the solid oxide fuel cell generates the minimum necessary power within the range according to power demand to reduce the exhaust fuel gas containing carbon dioxide and suppress the amount of carbon dioxide that becomes excessive in the system. The carbon dioxide stored in the storage device may be methanated by the methanation device when the hydrogen production amount of the hydrogen production device increases with the recovery of the power of the renewable energy power generation device to consume carbon dioxide without emitting carbon dioxide gas to the outside. Further, when the power of the renewable energy power generation device is insufficient for a long period of time, the external surplus power can be received to cover the power required to produce hydrogen. By utilizing carbon dioxide as a buffer for power supply in this way, it is possible to provide a function of adjusting the imbalance between renewable energy and surplus power and power demand. Further, when the power Pa of the renewable energy is significantly small relative to the supply amount Qs of the to-be-treated gas G, and carbon dioxide cannot be stored in the system, it is possible to take it out and use as a food material or an industrial gas without releasing it into the atmosphere.

(11) In another aspect, in any one of the above aspects (8) to (10), the system control unit is configured to, when the supply amount of the to-be-treated gas decreases and the power of the renewable energy power generation device increases, control the solid oxide fuel cell and the methanation device to decrease operating loads.

According to the above aspect (11), when the power of the renewable energy power generation device is sufficiently large relative to the supply amount of the to-be-treated gas, the hydrogen production device can supply hydrogen required to methanate all the carbon dioxide in the system. Therefore, all the carbon dioxide contained in the to-be-treated gas and in the exhaust fuel gas from the solid oxide fuel cell operated at low operating load can be methanated by the methanation device, so that no carbon dioxide is emitted to the outside.

(12) In another aspect, in any one of the above aspects (8) to (11), the system control unit is configured to, when the supply amount of the to-be-treated gas decreases and the power of the renewable energy power generation device decreases, supply methane from a methane gas supply source to the to-be-treated gas, control power generation amount of the solid oxide fuel cell according to power demand, and control the storage device to store excess carbon dioxide relative to production amount of the hydrogen produced by the hydrogen production device.

According to the above aspect (12), the solid oxide fuel cell generates power and thus discharges the exhaust fuel gas according to power demand, but since the power of the renewable energy power generation device is small, the hydrogen production device cannot provide sufficient hydrogen to methanate all the carbon dioxide in the system, resulting in excess carbon dioxide in the system. By storing such excess carbon dioxide in the system in the storage device, it is possible to prevent carbon dioxide emissions to the outside. The carbon dioxide stored in the storage device may be methanated by the methanation device when the hydrogen production amount of the hydrogen production device increases with the recovery of the power of the renewable energy power generation device to consume carbon dioxide without emitting carbon dioxide gas to the outside. Further, when the power Pa of the renewable energy is significantly small relative to the supply amount Qs of the to-be-treated gas G, and carbon dioxide cannot be stored in the system, it is possible to take it out and use as a food material or an industrial gas without releasing it into the atmosphere.

(13) In another aspect, in any one of the above aspects (1) to (12), the to-be-treated gas is a biogas discharged from a beer factory.

According to the above aspect (13), biogas discharged from a beer factory is used as the to-be-treated gas. In a beer factory, as the beer production amount greatly varies with the seasons, the emission amount of biogas greatly varies. By adjusting the operational balance of the components of the system, it is possible to achieve a clean power generation system that does not emit carbon dioxide while absorbing the variations in the supply amount of the to-be-treated gas in a buffering manner.

(14) In another aspect, in any one of the above aspects (1) to (12), the to-be-treated gas is a boil off-gas generated in a tank for storing a liquefied natural gas.

According to the above aspect (14), boil off-gas generated in a tank for storing a liquefied natural gas is used as the to-be-treated gas. The amount of boil off-gas varies depending on the environment around the tank and the remaining amount of the liquefied natural gas. By adjusting the operational balance of the components of the system, it is possible to achieve a clean power generation system that suppresses carbon dioxide emissions while absorbing the variations in the supply amount of the to-be-treated gas in a buffering manner.

### REFERENCE SIGNS LIST

100 Biogas-utilizing methanation system
102 To-be-treated gas supply line
104 Biogas supply source
106 Pretreatment device
108 Biogas supply line
110 City gas supply line
112 City gas supply source
113 City gas flow rate control valve
114 Solid oxide fuel cell
115 Power transmission circuit
116 Hydrogen production device
117 First exhaust fuel gas line
118 Methanation device
119 Dryer
120 Storage device
121 Water recovery device
122 System control unit
122a To-be-treated gas supply amount detection unit
122b Renewable energy power detection unit
122c Control unit
124 Produced methane supply line
130 Pure water tank
132 Recycle gas compressor
134 Carbon dioxide recovery device
135 Second exhaust fuel gas line
136 Carbon dioxide storage line
138 Storage gas supply line
139 Storage gas supply amount control valve
140 Hydrogen supply line
142 Methane purification device
144 Methane discharge line
145 Pure water supply line
146 Renewable energy power generation device
147 Pure water supply amount control valve
148 Oxidant supply line
150 Supply amount sensor
152 Power sensor

1. A biogas-utilizing methanation system, comprising:
a solid oxide fuel cell capable of generating power by using a to-be-treated gas containing methane and carbon dioxide as a fuel gas;
a hydrogen production device capable of producing hydrogen by using power of a renewable energy power generation device;
a methanation device capable of producing methane by methanation process using carbon dioxide contained in an exhaust fuel gas of the solid oxide fuel cell and the hydrogen produced by the hydrogen production device, and yielding the methane as a chemical raw material or supplying the methane to the solid oxide fuel cell as the fuel gas;
a methane purification device capable of purifying a methane gas produced by the methanation device, and supplying at least part of the methane to outside as a chemical raw material and supplying an off-gas to the solid oxide fuel cell; and
a storage device capable of storing at least part of the carbon dioxide supplied to the methanation device on the basis of at least one of supply amount of the to-be-treated gas or the power of the renewable energy power generation device.

2. A biogas-utilizing methanation system, comprising:
a hydrogen production device capable of producing hydrogen by using power of a renewable energy power generation device;
a methanation device capable of producing methane by methanation process using a to-be-treated gas containing methane and carbon dioxide and the hydrogen produced by the hydrogen production device;
a methane purification device capable of purifying the methane produced by the methanation device, and supplying at least part of the methane to outside as a chemical raw material and supplying an off-gas to a solid oxide fuel cell;
the solid oxide fuel cell capable of generating power by using the off-gas of the methane purification device; and
a storage device capable of storing at least part of the carbon dioxide supplied to the methanation device on the basis of at least one of supply amount of the to-be-treated gas or power of the renewable energy power generation device.

3. The biogas-utilizing methanation system according to claim 1,
wherein the storage device is capable of storing carbon dioxide recovered from an exhaust fuel gas of the solid oxide fuel cell, is connected to the methanation device via a storage gas supply line provided with a flow rate control valve, and is capable of supplying carbon dioxide to outside as a food raw material or an industrial gas.

4. The biogas-utilizing methanation system according to claim 1,
wherein the biogas-utilizing methanation system is configured to supply the power generated by the solid oxide fuel cell and surplus power to the hydrogen production device when the power of the renewable energy power generation device is insufficient.

5. The biogas-utilizing methanation system according to claim 1,
wherein the biogas-utilizing methanation system is configured to supply oxygen produced by the hydrogen production device as part of an oxidizing gas of the solid oxide fuel cell.

6. The biogas-utilizing methanation system according to claim 1, further comprising a methane supply source capable of supplying methane to the to-be-treated gas.

7. The biogas-utilizing methanation system according to claim 1,
wherein the solid oxide fuel cell is capable of discharging an exhaust fuel and an exhaust oxidizing gas independently.

8. The biogas-utilizing methanation system according to claim 1, further comprising a system control unit for controlling at least part of the solid oxide fuel cell, the hydrogen production device, the methanation device, or the storage device, on the basis of at least one of the supply amount of the to-be-treated gas or the power of the renewable energy power generation device.

9. The biogas-utilizing methanation system according to claim 8,
wherein the system control unit is configured to, when the supply amount of the to-be-treated gas increases and the power of the renewable energy power generation device increases, control the solid oxide fuel cell according to fluctuation amount of the power, and control the methanation device to increase an operating load.

10. The biogas-utilizing methanation system according to claim 8,
wherein the system control unit is configured to, when the supply amount of the to-be-treated gas increases and the power of the renewable energy power generation device decreases, control power generation amount of the solid oxide fuel cell according to power demand, control the storage device to store excess carbon dioxide relative to production amount of the hydrogen produced by the hydrogen production device, and use surplus power to ensure that storage amount of carbon dioxide is not excessive.

11. The biogas-utilizing methanation system according to claim 8,
wherein the system control unit is configured to, when the supply amount of the to-be-treated gas decreases and the power of the renewable energy power generation device increases, control the solid oxide fuel cell and the methanation device to decrease operating loads.

12. The biogas-utilizing methanation system according to claim 8,
wherein the system control unit is configured to, when the supply amount of the to-be-treated gas decreases and the power of the renewable energy power generation device decreases, supply methane from a methane gas supply source to the to-be-treated gas, control power generation amount of the solid oxide fuel cell according to power demand, and control the storage device to store excess carbon dioxide relative to production amount of the hydrogen produced by the hydrogen production device.

13. The biogas-utilizing methanation system according to claim 1,
wherein the to-be-treated gas is a biogas discharged from a beer factory.

14. The biogas-utilizing methanation system according to claim 1,
wherein the to-be-treated gas is a boil off-gas generated in a tank for storing a liquefied natural gas.
